(19) United States
(12) Patent Application Publication
(10) Pub. No.: US 2003/0118601 A1
(43) Pub. Date: Jun. 26, 2003

(54) FIV vaccine

(76) Inventors: James Charles Neil, Glasgow (GB); Mark Alan Rigby, Glasgow (GB); James Oswald Jarrett, Milngavie (GB)

Correspondence Address: MYERS BIGEL SIBLEY & SAJOVEC, PO BOX 37428, RALEIGH, NC, 27627, US

(21) Appl. No.: 10/205,534

(22) Filed: Jul. 25, 2002

Related U.S. Application Data
(63) Continuation of application No. 09/380,990, filed on Nov. 8, 1999, now abandoned.
Application No. 09/380,990 is a 371 of international application No. PCT/GB98/00715, filed on Mar. 10, 1998.

(30) Foreign Application Priority Data
Mar. 11, 1997 (GB) 9704977.9

(51) Int. Cl. A61K 48/00; A61K 38/21; A61K 39/12

(52) U.S. Cl. 424/186.1; 514/44; 424/85.5

(57) ABSTRACT

Vaccine formulations for FIV related disease comprising a FIPV polynucleotide comprising a dysfunctional pol gene, FIPV polynucleotide fragments, and uses therefor in the prophylaxis and/or treatment of FIV-related diseases.

### BACKGROUND

The present invention relates to a feline immunodeficiency proviral (FIPV) polynucleotide fragment comprising a dysfunctional pol gene region, a recombinant vector comprising said FIVP polynucleotide fragment, a host cell containing said FIPV polynucleotide fragment, a feline immunodeficiency virus (FIV) vaccine comprising said FIPV polynucleotide fragment, a method of treating FIV-related disease, and pharmaceutical compositions comprising said FIPV polynucleotide fragment for use as a prophylactic and/or therapeutic agent in cats.

Feline immunodeficiency virus (FIV) is a member of the Retroviridae; it is a lentivirus which is associated with a debilitating immunodeficiency syndrome in cats (Pedersen N. C. et al., Science (1987) Vol. 235, pp. 790-793).

Lentiviruses by nature do display a large degree of molecular and biological variation. This natural variation is thought to be in part ascribable to the low fidelity of the viral enzyme reverse transcriptase in the process of copying the viral genomic RNA to DNA (Preston et al., Science 242: 1168-1171 (1988), Roberts et al., Science 242: 1171-1173 (1988)). As a result, several variant FIV-strains have been found.

To date, isolates of several variant FIV strains, some of which have been subjected to molecular cloning, have been described. Amongst these strains are two isolates from the United States (Petaluma-strains (Olmsted et al., Proc. Natl. Acad. Sci USA 86: 8088-8092 (1989), Talbott et al., Proc. Natl. Acad. Sci. USA 86: 5743-5747 (1989)) and San Diego strain (Phillips et al., J. Virol. 64: 4605-4613 (1990)), one from the United Kingdom (Harbour et al., Vet. Rec. 122: 84-86 (1988)) and two from Japan (Ishida et al., J. Am. Vet. Med. Assoc. 194: 221-225 (1989), Miyazawa et al., Arch. Virol. 108: 59-68 (1989)), which were obtained from the DNA of in vitro propagated strains. One strain, the F14 clone of Olmsted et al., supra has been deposited in the Genbank data base under Accession No. M25381.

Molecular characterisation and determination of heterogeneity between FIV isolates has been described by Maki et al., (Arch. Virol. 123: 29-45 (1992)). The construction of DNA clones from two FIV proteins, i.e. the envelope (ENV) protein and the virion core (GAG) protein and their use for detecting and preventing FIV has been described in WO 92/15684.

Sero-epidemiological surveys have revealed that the virus occurs all over the world (Furuya et al., Jpn. J. Vet. Sci. 52: 891-893 (1990), Gruffydd-Jones et al., Vet. Rec. 123: 569-570, (1988), Ishida et al., Jpn. J. Vet. Sci. 52: 453-454 (1990), Ishida et al., Japn. J. Vet. Sci. 50: 39-44 (1988), Ishida et al., J. AM. Vet. Med. Assoc. 194: 221-225 (1989), Swinney et al., N. Z. Vet. J. 37: 41-43 (1989)).

FIV has a complex genome structure comprising group antigen proteins (GAG), which are the major structural proteins of the virus; POL, proteins of the polymerase gene; and ENV, proteins of the envelope gene. The gag gene encodes matrix, capsid and nucleocapsid proteins, and the pol gene encodes protease, reverse transcriptase, dUTPase and integrase. The env gene encodes surface and transmembrane envelope glycoproteins. In addition to the structural and enzymatic proteins, at least three more genes (Vif, ORFA, Rev) are present in FIV (Miyazawa T., Arch. Virol. (1994) Vol. 134 pp. 221-234). As with other members of the Retroviridae, the integrated genome of FIV is bordered by long terminal repeats (LTRs) comprised of U5, R, and U3 domains. Likewise, the basic structural elements gag, pol and env are encoded in the approximate 9500 base pair genome. In addition to these common elements, FIV encodes several short open reading frames (sORFs). Details of the genomic organisation of FIV may be found in “Infectious Agents and Disease Vol. 2 pp. 361-374 (1994)” under the review paper by John H. Elder and Tom R. Phillips.

Control by vaccination of FIV infection has been a long-sought goal.

WO 94/20622 describes the provision of a vaccine against FIV comprising a polypeptide fragment of an FIV surface protein which is capable of inducing neutralising antibodies against FIV. There is no reference to the potential or actual use of proviral FIV DNA in the production of DNA vaccines against FIV infection.

Development of protective FIV vaccines has proven difficult (Hosie M. J. and Yamamoto J. K. (1995) Feline Immunology and Immunodeficiency (Willett B. J. and Jarrett O. Eds.) Oxford University Press, New York, pp. 263-278). An initial success was reported with the development of a cell line (FL4) that constitutively releases large numbers of FIV particles (Yamamoto J. K. et al. (1991) Inter-virology Vol. 32, pp. 361-375). Inactivated viral and whole cell vaccines based on this cell line showed the first evidence of protection against FIV infection, however, this protection has subsequently been shown to be of limited spectrum (Hosie M. J. et al., (1995) J. Virol. 69 pp. 1253-1255), suggesting that the reported strategy will be less useful for antigenically diverse natural isolates of FIV that are not readily propagated in vitro. Subunit vaccines for FIV have not been particularly successful to date. While viral load reduction after challenge has been demonstrated in animals immunised with glycoprotein purified from virions (Hosie M. J. et al., (1996) Vaccine Vol. 14 pp. 405-411), studies using recombinant proteins as immunogens led instead to enhancement of early infection (Hosie M. J. et al., (1992) Vet. Immunol. Pathol. Vol. 35, pp. 191-198; Siebelink K. H. J. et al., (1995) J. Virol. Vol. 69, pp. 3704-3711).

Genetic immunisation for eliciting an immune response was first reported by Tang D. C. et al., (1992) Nature (London) Vol. 356, pp. 152-154. A general review on genetic immunisation is further reported by Hassett D. E. and Whitton J. L. in Trends. Microbiol. (1996) Vol. 4, pp. 307-312. Protective immunisation has been achieved in virus-host systems using inoculation of DNA (Fynan E. F. et al., (1993) Proc. Natl. Acad. Sci. USA Vol. 90, pp. 11478-11482; Webster R. G. et al., (1994) Vaccine Vol. 12, pp. 1495-1498). However, efforts so far have employed plasmids containing individual viral genes or combinations of genes but have been restricted to non-replicating vectors. Protection against infection by lentiviruses such as FIV has been attempted by expression of the ENV protein of FIV in cats (Cuisinier A-M et al., (1996) 3rd International Feline Retrovirus Research Symposium, Fort Collins, Colo.).

The above outlined problems emphasise the need to consider alternative and innovative approaches to lentivirus vaccination and in particular, FIV vaccination.

The prior art does not teach the use of FIV pol region deletion mutants comprising a dysfunctional reverse transcriptase (RT) gene region in the manufacture and use of vaccines against FIV related disease.

It is thought that DNA delivery may improve the prospects for the use of attenuated viral vaccines, since it may be possible to deliver more comprehensively disabled viral derivatives that cannot be obtained as stable high-titer viruses.

The present invention seeks to mitigate against the disadvantages associated with the prior art.

According to a first aspect of the invention there is provided a vaccine formulation comprising a feline immunodeficiency provirus (FIPV) polynucleotide comprising a dysfunctional pol gene which is substantially incapable of encoding a functionally competent reverse transcriptase (RT) or a functional RT fragment thereof.

A “FIPV” polynucleotide can be viewed as a polynucleotide fragment of an FIV capable of integration into a host cell genome. Host cells comprising FIPV of the invention are capable of producing FIV proteins, except for functionally competent RT or functionally competent fragments thereof. As such, host cells for the FIPV of the invention are able to release non-infectious FIV viral particles i.e. FIV particles which are substantially incapable of replication.

A “dysfunctional pol gene” is one which is substantially incapable of coding for a native RT or a functional equivalent thereof. Thus a “dysfunctional pol gene” means that the pol gene has been modified by an in-frame deletion, insertion or substitution (or other change in the DNA sequence such as rearrangement) such that the pol gene is generally unable to express a functionally competent RT or a functionally competent equivalent polypeptide product thereof.

pol genes of the invention which are substantially incapable of encoding a functionally competent RT may be rendered dysfunctional by any one of several ways:

(i) A deletion of the entire in-frame RT coding domain of the pol gene from a wild type FIPV genome. For example, depending on the wild type of FIPV or FIV of concern, a deletion of the nucleotide sequence from a wild type FIPV or FIV genome between about nucleotide 2337±12 bases to about nucleotide 4013±12 bases can be made. An example of a FIV clone from which a deletion can be made is the F14 clone of FIV. Using this clone a deletion of the entire in-frame RT coding region can be made between nucleotide 2337 and nucleotide 4013. The in-frame deletion should be such so as not to substantially affect the expression of other gene products from the FIV or FIPV genome.

(ii) A deletion of a portion of the in-frame RT coding domain of the pol gene of a wild type FIPV genome. A “portion of the in-frame RT coding domain” means a polynucleotide fragment which by its deletion from the RT coding region is sufficient to render any RT or fragment or fragments thereof encoded and/or expressible thereby, substantially incapable of a physiological activity attributable to that of a functional RT produced by a FIV or FIPV. The deletion portion of RT may comprise a deletion of a small number of nucleotides, for example, 1, 2 or more nucleotides. Such deletions within the RT encoding domain of the pol gene can be achieved using recombinant DNA technology. Thus, the translational ORF for an RT can be altered resulting in the production of a protein which lacks the physiological functionality or functional competence of an RT found under native circumstances, for example, an RT derived from a pol gene in a wild type FIPV or FIV. The skilled addressee will also appreciate that such deletions in the translational ORF of the RT domain of the pol gene may also give rise to a dysfunctional pol gene which is substantially incapable of coding for a functionally competent RT, truncated RT even any RT or polypeptide fragment thereof. Such proteins/polypeptides, if produced, generally lack the functional competence typical of the enzyme, RT.

(iii) The deletion of the or a portion of the RT domain of the pol gene as described in (i) or (ii) above will leave a “gap” in the pol gene. A suitable polynucleotide fragment, such as a gene or gene fragment or genes or fragments thereof may be inserted into the “gap”. Gene insertions can include genes which express polypeptides capable of augmenting an immune response, such as feline cytokines, for example, γ feline interferon or other genes such as marker genes. Suitable marker genes may include but are not restricted to enzyme marker genes, for example the lac-Z gene from E. coli, antibiotic marker genes such as hygromycin, neomycin and the like. Generally, marker genes, if any, may be employed in an RT deletion. FIPV or FIV mutants of the invention should be such so as to not cause substantial deleterious or long lasting side-effects to a recipient animal.

In a preferment, the “gap” made by the deletion of the or a portion of the RT domain of the pol gene from a FIPV is not filled with a polynucleotide insert, the cut ends of the deletion site being ligated together using conventional recombinant DNA technology. The skilled addressee will also appreciate that the “gap” left by the partial or total deletion of the RT encoding region of the pol gene may be filled with a polynucleotide sequence which is a nonsense nucleotide sequence or an anti-sense sequence: In both instances any defective RT which may be produced from a polynucleotide fragment including such sequences should be incapable of RT functionality.

(iv) Nucleotide insertions can also be made at suitable restriction enzyme sites within the RT coding region using recombinant DNA technology. Such insertions can give rise to a dysfunctional RT or fragment(s) thereof which are substantially incapable of an RT activity. For example, when using the FIV F14 clone, stop codons may be inserted into the RT region at suitable insertion sites such as at the Pac 1 restriction site (nucleotide 3540 to 3547) of the RT encoding region of the pol gene, which can result in the production of a non-functional fragment(s) of RT.

A “functionally competent reverse transcriptase” is one which is capable of RT functionality. That is to say, an RT functionality permitting the copying of a ribose nucleic acid to a deoxyribose nucleic acid form, for example, in a host cell or in the genome of a host organism such as a feline. Thus, FIPV's of the invention comprising dysfunctional pol genes are substantially incapable of giving rise to infectious FIV particles.

As a preferment, there is provided a vaccine formulation wherein the FIPV polynucleotide comprises a deletion, still preferably an in-frame deletion, within the RT domain of the pol gene.

In a preferment there is provided a defective FIPV polynucleotide fragment comprising an in-frame deletion and/or insertion comprising at least one nucleotide in the RT region within the RT domain of the pol gene. The deletion should be such that coding sequences for other gene products of the FIPV, for example the pol gene products and other FIPV gene products, upstream and/or downstream from the RT domain are not substantially affected. That is to say that other gene products ordinarily having an immunogenic function and which are expressed from the FIPV substantially retain their immunogenic function. The deletion may be made between about nucleotide 2337±12 bases and 4013±12 bases of the RT domain of the pol gene depending on the FIV isolated. The deletion can be of any size so long as any RT polypeptide product which may be generated, such as an RT fragment thereof (or RT fragments thereof) does (do) not possess RT functionality and any coding sequences upstream or downstream thereof are not substantially affected. The deletion can be made starting at any suitable restriction enzyme site located in the RT region of the pol gene. However, it is preferred if the deletion is made starting at a restriction site which is unique to within the RT domain of the pol gene, if not the whole FIPV such as Nco1, Pac 1 and Sph 1. A suitable example of a starting restriction enzyme site, thought to be unique to at least within the RT region of the FIV F14 clone is the Pac 1 site located at nucleotides 3540-3547 thereof. The skilled addressee will appreciate that other FIV or FIPV isolates comprising similar enzyme restriction sites within the RT domain of the pol gene are encompassed by the present invention.

In a preferment there is provided a defective FIPV comprising a polynucleotide fragment deletion in the RT domain of the pol gene wherein the deletion is from nucleotide 3497 to nucleotide 3595 of the RT domain.

In a further embodiment of the invention, the defective FIVP can form part of a recombinant nucleic acid molecule comprising a replication defective FIPV under the control of regulatory sequences which enable expression of viral gene products in a host cell genome and production of FIV proteins other than functional RT or functional fragments thereof.

Regulatory sequences enabling integration and/or production of FIV proteins other than functional RT or functional fragments thereof can be promoter sequences which may or may not be associated with appropriate enhancer sequences. Suitable promoters include those as outlined by Norimine J. et al., (1992) J. Vet. Med. Sci. 51(1) pp. 189-191, and may include promoters obtained or derived from prokaryotic, eucaryotic and/or viral origins. Examples of promoters include but are not limited to the cytomegalovirus (CMV) promoter immediate early (IE) promoter region, for example the human cytomegalovirus (HCMV) immediate early (IE) promoter region, the Rous sarcoma virus (RSV) long terminal repeat (LTR), feline leukaemia virus (FeLV) LTR, simian immunodeficiency virus from African green monkey (SIV AGM) LTR, and the SV40 early-promoter region.

The person skilled in the art will also appreciate that the natural promoter sequence of the defective FIPV carrying a dysfunctional pol gene (i.e. located in the 5′ LTR thereof) could also form part of a recombinant nucleic acid molecule of the invention.

Thus, FIPV of the invention can be obtained by taking cDNA encompassing the genome of an appropriate FIV isolate and inserting it into a suitable vector, such as a pGEM vector or a lambda vector. A suitable FIV clone is the F14 clone of FIV-Petaluma described by Olmsted R. A. et al. (1989) Proc. Natl. Acad. Sci. (USA) Vol. 86 pp. 8088-8092. The FIV clone can then be linearised using an appropriate restriction enzyme such as Nco 1, Sph 1, Bae 1 Pac 1 and the like, the linearised vector is then purified, for example by precipitation followed by digestion with a suitable exonuclease such as Bal31under appropriate exonuclease digestion conditions for a desired period of time (Maniatis et al. Molecular Cloning—a Laboratory Manual; Cold Spring Harbor Laboratory Press First Edition (1989) p 135). After further purification, suitably by organicsolvent extraction and alcohol precipitation, appropriately exonuclease digested nucleic acid molecules can be re-circularised by ligation and the products thereof used to transform an appropriate host cell, such as a bacterium host cell, e.g. E.coli. Clones thus obtained may then be characterised by polymerase chain reaction (PCR) amplification across the nucleic acid molecule in order to ascertain the size and location of the deletion in the RT domain of the pol gene (i.e. in-frame or otherwise).

A suitably sized deletion region has been found to be a 235 bp region of the pol gene of the FIV Petaluma strain within which is found the Pac 1 restriction enzyme site.

The deletion generally has to be made in the RT domain of the pol gene in a position such that any defective FIPV incorporated into a host cell genome retains a sufficient immunogenic function to elicit, on expression of protein or polypeptides encoded by the FIPV, at least a cellular immune response (such as a cytotoxic T-cell response) in a host animal, such as a feline.

Suitable clones comprising deletion regions of the invention can be further characterised using DNA sequence analysis using primers of any acceptable length, such as primers of up to 60 nucleotide bases in length, preferably primers of about 20 to 60 nucleotide bases in length. More preferably such primers are from 20 to 30 nucleotides in length.

The selection of vector is not critical provided that it is able to carry the desired FIV clone into a suitable host cell. The host cell can be one in which replication of the recombinant vector molecule can occur. The host cell can be a cell in which regulatory sequences of the or at least one other vector can also be recognised such that at least a further polypeptide fragment(s), such as a fragment capable of augmenting or eliciting at least an immune response as described above, can be expressed. For example, if the prophylactic and/or therapeutic effect of an appropriately cloned FIPV of the present invention is to be augmented, a further vector encoding an appropriate adjuvant protein or polypeptide, such as a cytokine coding vector, for example, a feline γ interferon (γIFN) coding vector, can also be employed as a component of a vaccine or pharmaceutical composition of the invention. International Patent Application WO 96/03435 describes the provision of a feline γ interferon, and includes the provision of a polynucleotide fragment encoding feline γ interferon and vectors therefor. Such polynucleotide fragments as described in WO 96/03435 can be administered in conjunction with vectors coding for defective FIPV of the invention to animals in need thereof.

A wide range of vectors is currently known, including vectors for use in bacteria, e.g. pBR322, 325 and 328, various pUC-vectors a.o. PUC 8, 9, 18, 19, specific expression-vectors; PGEM, pGEX, and Bluescript®, vectors based on bacteriophages; lambda-gtWes, Charon 28, M13-derived phages, vectors containing viral sequences on the basis of SV40, papilloma-virus, adenovirus or polyomavirus (Rodriquez, R. L. and Denhardt, D. T., ed.; Vectors: A survey of molecular cloning vectors and their uses, Butterworths (1988), Lenstra et al., Arch. Virol.; 110: 1-24 (1990)).

All recombinant molecules comprising the nucleic acid molecule under the control of regulatory sequences enabling expression of the defective FIPV by said nucleic acid molecule are considered to be part of the present invention.

Thus, as a further embodiment of the invention there is provided a vector comprising a defective FIPV in recombinant form under the control of regulatory sequences enabling expression of viral proteins of the FIPV yet which is substantially unable to express a functional RT or a functional fragment thereof.

In a further embodiment of the invention there is provided a host cell comprising a dysfunctional FIVP or the present invention under the control of a regulatory sequence enabling expression of viral proteins of the FIPV yet which is substantially unable to express a functional RT or a functional fragment thereof.

A host cell may be a cell of bacterial origin, e.g. Escherichia coli, Bacillus subtilus and Lactobacillus species, in combination with bacteria-based vectors as PBR322, or bacterial expression vectors as pGEX, or with bacteriophages. The host cell may also be of eukaryotic origin, e.g. yeast-cells in combination with yeast-specific vector molecules, or higher eukaryotic cells such as insect cells (Luckow et al; Bio-technology 6: 47-55 (1988)) in combination with vectors or recombinant baculoviruses, plant cells in combination with e.g. Ti-plasmid based vectors or plant viral vectors (Barton, K. A. et al; Cell 32: 1033 (1983), cells of mammalian origin such as Hela cells, Chinese Hamster Ovary cell (CHO) or Crandell Feline Kidney-cells, also with appropriate vectors or recombinant viruses.

The FIPV fragment according to the present invention may be cloned under the control of a promoter sequence or not under the control of a promoter sequence in a viral genome, as the case may be. In such a manner, the virus may be used as a means of transporting the FIPV fragment into a target cell. Such recombinant viruses are called vector viruses. The site of integration may be a site in a gene not essential to the virus, or a site in an intergenic region. Viruses often used as vectors are Vaccinia viruses (Panicali et al; Proc. Natl. Acad. Sci. USA, 79: 4927 (1982), Herpesviruses (E.P.A. 0473210A2), Retroviruses (Valerio, D. et al; in Baum, S. J., Dicke K. A., Lotzova, E. and Pluznik, D. H. (Eds.), Experimental Haematology today—1988. Springer Verlag, N.Y.: pp 92-99 (1989)) and baculoviruses (Luckow et al; Bio-technology 6: 47-55 (1988)).

The invention also comprises a virus vector containing a FIPV fragment or a recombinant nucleic acid molecule encoding the FIPV fragment under the control of regulating sequences enabling expression of the protein encoded by said nucleic acid sequence.

In an alternative, defective FIPV polynucleotides of the invention may be applied directly to the cells of an animal in vivo, or by in vitro transfection of cells taken from the said animal, which cells are then introduced back into the animal. Defective FIPV may be delivered to various cells of the animal body including muscle, skin or blood cells thereof. The defective FIPV may be loaded for example, into muscle or skin using a suitable loading means such as a syringe. Methods of applying naked defective FIPV of the invention directly to thebody are described in WO 90/11092, especially at pages 35 to 43 thereof.

As such, defective FIPV polynucleotides of the invention may be administered as pharmaceutically acceptable salts to animals in need thereof.

Polynucleotide salts: Administration of pharmaceutically acceptable salts of the polynucleotides described herein is included within the scope of the invention. Such salts may be prepared from pharmaceutically acceptable non-toxic bases including organic bases and inorganic bases. Salts derived from inorganic bases include sodium, potassium, lithium, ammonium, calcium, magnesium, and the like. Salts derived from pharmaceutically acceptable organic non-toxic bases include salts of primary, secondary, and tertiary amines, basic amino acids, and the like. Further pharmaceutical salts are described in, S. M. Berge et al., Journal of Pharmaceutical Sciences 66: 1-19 (1977).

Polynucleotides for injection, may be prepared in unit dosage form in ampules, or in multidose containers. The polynucleotides may be present in such forms as suspensions, solutions, or emulsions in oily or preferably aqueous vehicles. Alternatively, the polynucleotide salt may be in lyophilized form for reconstitution, at the time of delivery, with a suitable vehicle, such as sterile pyrogen-free water. Both liquid as well as lyophilized forms that are to be reconstituted will comprise agents, preferably buffers, in amounts necessary to suitably adjust the pH of the injected solution. For any parenteral use, particularly if the formulation is to be administered intravenously, the total concentration of solutes should be controlled to make the preparation isotonic, hypotonic, or weakly hypertonic. Nonionic materials, such as sugars, are preferred for adjusting tonicity, and sucrose is particularly preferred. Any of these forms may further comprise suitable formulatory agents, such as starch or sugar, glycerol or saline. The compositions per unit dosage, whether liquid or solid, may contain from 0.1% to 99% of polynucleotide material.

In a further embodiment of the invention there is provided a vaccine against FIV comprising a defective FIPV polynucleotide fragment of the invention. The FIPV fragment may take the form of a naked FIPV polynucleotide fragment, that is, a FIPV polynucleotide fragment not bound up in a vector form, such as a plasmid form. The vaccine of the invention may optionally include a further polynucleotide fragment encoding a further compound having an immunogenic function such as a cytokine, for example, feline γ interferon. The additional polynucleotide fragment may be in the form of a further vector as described herein, for example an additional plasmid vector. Alternatively, the additional polynucleotide can be in the form of a naked DNA. Such naked DNA may be adhered to a microprojectile or in an appropriate holding solution, such as a saline solution. Alternatively, the FIPV polynucleotide fragment can be available in the form of a vector or of a host cell.

The vaccine may also comprise a dysfunctional FIPV polynucleotide fragment as described hereinbefore in combination with a further vector or further polynucleotide fragment encoding a gene which when expressed the gene product thereof retains an immunogenic function. A suitable further polynucleotide fragment for use in a vaccine of the invention can be selected from those described in WO 96/03435, such as vectors encoding feline γ interferon.

In a preferred presentation, the vaccine can also comprise an adjuvant. Adjuvants in general comprise substances that boost the immune response of the host in a non-specific manner. A number of different adjuvants are known in the art. Examples of adjuvants may include Freund's complete adjuvant, Freund's Incomplete adjuvant, liposomes, and niosomes as described in WO 90/11092, mineral and non-mineral oil-based water-in-oil emulsion adjuvants, cytokines, short immunostimulatory polynucleotide sequences, for example in plasmid DNA containing CpG dinucleotides such as those described by Sato Y. et al. (1996) Science Vol. 273 pp. 352-354; Krieg A. M. (1996) Trends in Microbiol. 4 pp. 73-77. Further adjuvants of use in the invention include encapsulators comprising agents capable of forming microspheres (1-10 82 m) such as poly(lactide-coglycolide), facilitating agents which are capable of interacting with polynucleotides such that the said polynucleotide is protected from degradation and which agents facilitate entry of polynucleotides such as DNA into cells. Suitable facilitating agents include cationic lipid vectors such as:

1,3-di-oleoyloxy-2-(6-carboxy-spermyl)-propylamid (DOSPER),

N-[1-(2,3-dioleoyloxy)propyl]-N,N,N-trimethylammoniummethylsulfate (DOTAP),

N-[1-(2,3-dioleoyloxy)propyl)]-N,N,N-trimethylammonium chloride (DOTMA),

(N,N,N′,N′-tetramethyl-N,N′-bis(2-hydroxylethyl)-2,3-dioleoyloxy-1,4-butanediammonium iodide,

bupivacaine-HCl,

non-ionic polyoxypropylene/polyoxyethylene block copolymers,

polyvinyl polymers and the like.

Such cationic lipid vectors can be combined with further agents such as L-dioleoyl phosphatidyl ethanolamine (DOPE) to form multilamellar vesicles such as liposomes.

The vaccine may also comprise a so-called “vehicle”. A vehicle is a compound, or substrate to which the FIPV polynucleotide fragment can adhere, without being covalently bound thereto. Typical “vehicle” compounds include gold particles, silica particles such as glass and the like. Thus FIPV polynucleotides of the invention can be introduced into appropriate cells using biolistic methods such as the high-velocity bombardment method using polynucleotide coated gold particles as described in the art (Williams R. S. et al. (1991) Proc. Natl. Acad. Sci. USA 88 pp. 2726-2730; Fynan E. F. et al. (1993) Proc. Natl. Acad Sci. USA Vol. 90 pp. 11478-11482).

In addition, the vaccine may comprise one or more suitable surface-active compounds or emulsifiers, e.g. Span or Tween.

In a further aspect of the invention there is provided the use of a FIPV polynucleotide fragment as described herein for producing at least a cell mediated immunity to FIV which comprises a defective FIPV as described above for the manufacture of a FIV vaccine for the prophylaxis and/or treatment of FIV-related disease. Preferably, there is provided use of a FIPV polynucleotide fragment in naked or vector form for the manufacture of a FIV vaccine for the prophylaxis and/or treatment of FIV infection. Most preferably, the use is in felines.

In a further aspect of the invention there is provided a method of treating animals which comprises administering thereto a vaccine composition comprising a defective FIPV polynucleotide fragment as described herein to animals in need thereof. Preferably, the animals are felines. Naturally, the vaccine formulation may be formulated for administration by oral dosage (e.g. as an enteric coated tablet), by parenteral injection or otherwise.

The invention also provides a process for preparing a FIV virus vaccine, which process comprises admixing a defective FIVP polynucleotide fragment in naked or vector form as herein described with a suitable carrier or adjuvant.

The mode of administration of the vaccine of the invention may be by any suitable route which delivers an immunoprotective amount of the virus of the invention to the subject. However, the vaccine is preferably administered parenterally via the intramuscular or deep subcutaneous routes. Other modes of administration may also be employed, where desired, such as oral administration or via other parenteral routes, i.e., intradermally, intranasally, or intravenously.

Generally, the vaccine will usually be presented as a pharmaceutical formulation including a carrier or excipient, for example an injectable carrier such as saline or a pyrogenic water. The formulation may be prepared by conventional means.

It will be understood, however, that the specific dose level for any particular recipient animal will depend upon a variety of factors including age, general health, and sex; the time of administration; the route of administration; synergistic effects with any other drugs being administered; and the degree of protection being sought. Of course, the administration can be repeated at suitable intervals if necessary.

As a further aspect of the invention there is provided a polynucleotide fragment encoding for an FIPV which is substantially incapable of encoding a functional RT or a functional RT fragment thereof for use as a medicament for FIV-related disease. The skilled addressee will appreciate that a deletion may be made in the RT domain of the pol gene which deletion may be an in-frame deletion as described herein. The skilled addressee will also appreciate that insertions into deletion sites may be made to FIPV of the invention as utilised under this aspect of the invention as described herein.

As a further aspect of the invention there is provided use of an FIPV comprising a dysfunctional pol gene in the manufacture of a vaccine for the prophylaxis and/or therapy of FIV-related disease. In a preferment the pol gene comprises a deletion within its RT domain, such as an in-frame deletion as described herein. The skilled addressee will also appreciate that insertions into deletion sites may be made to FIPV of the invention as utilised under this aspect of the invention as described herein.

Embodiments of the invention will now be illustrated by way of the following Figures and Examples.

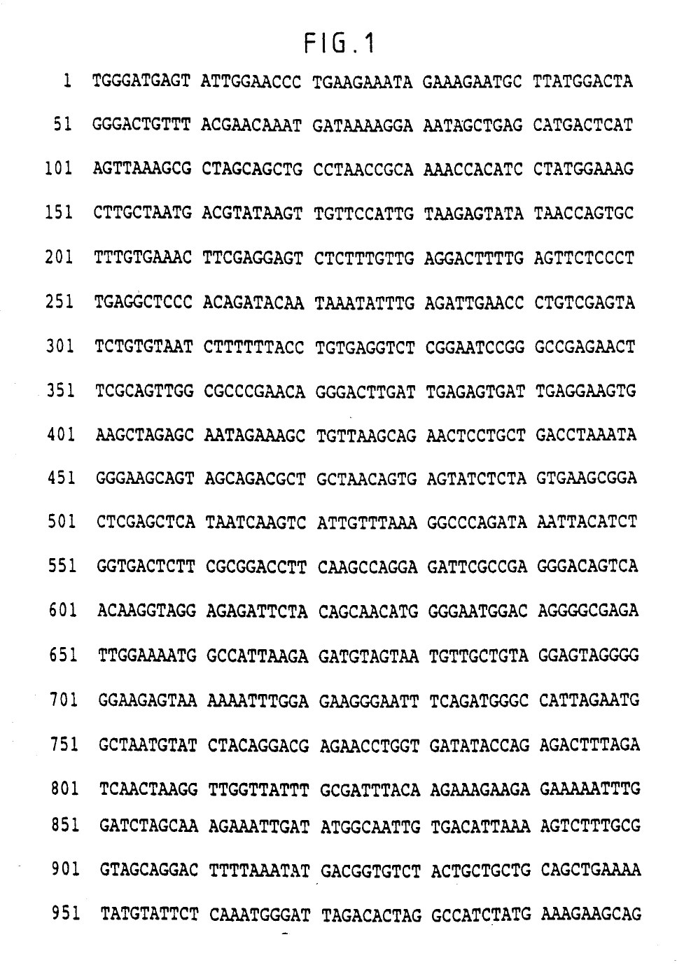

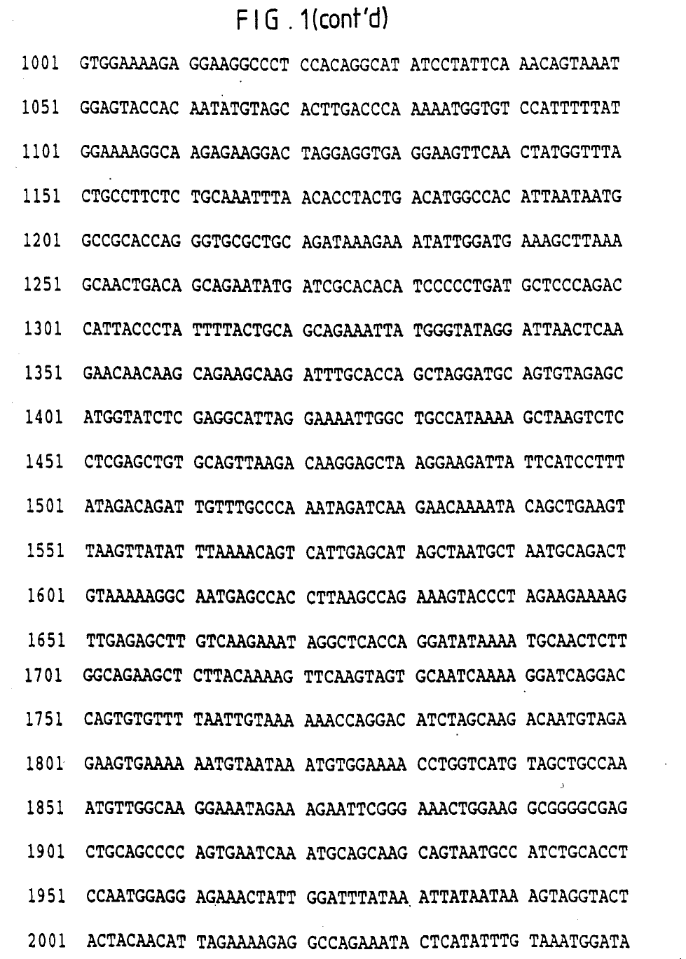

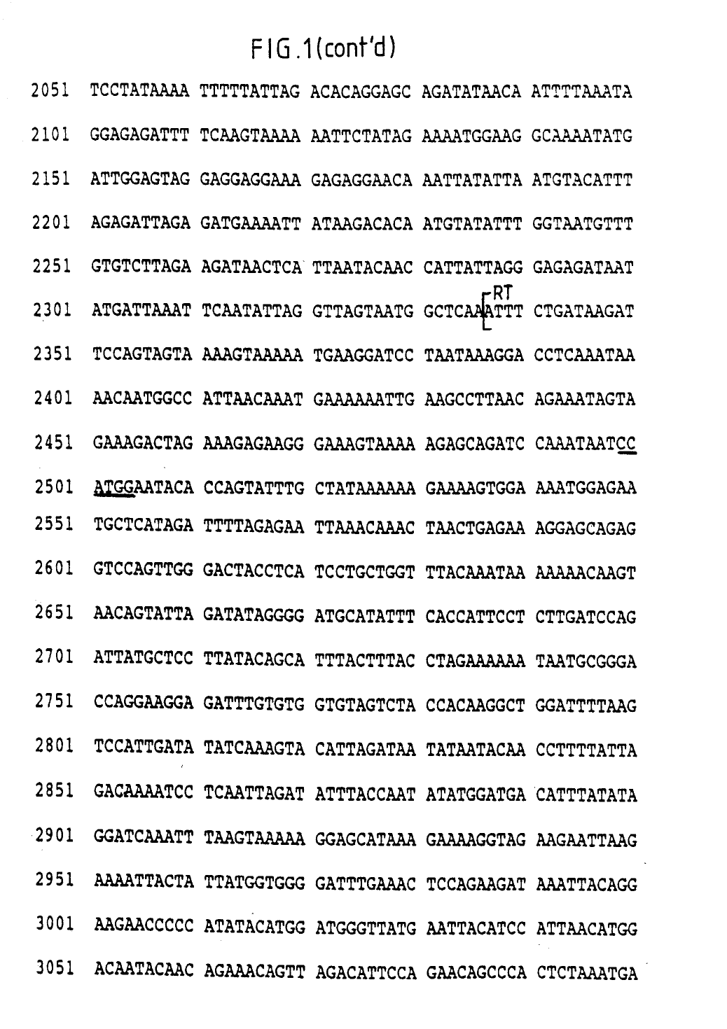

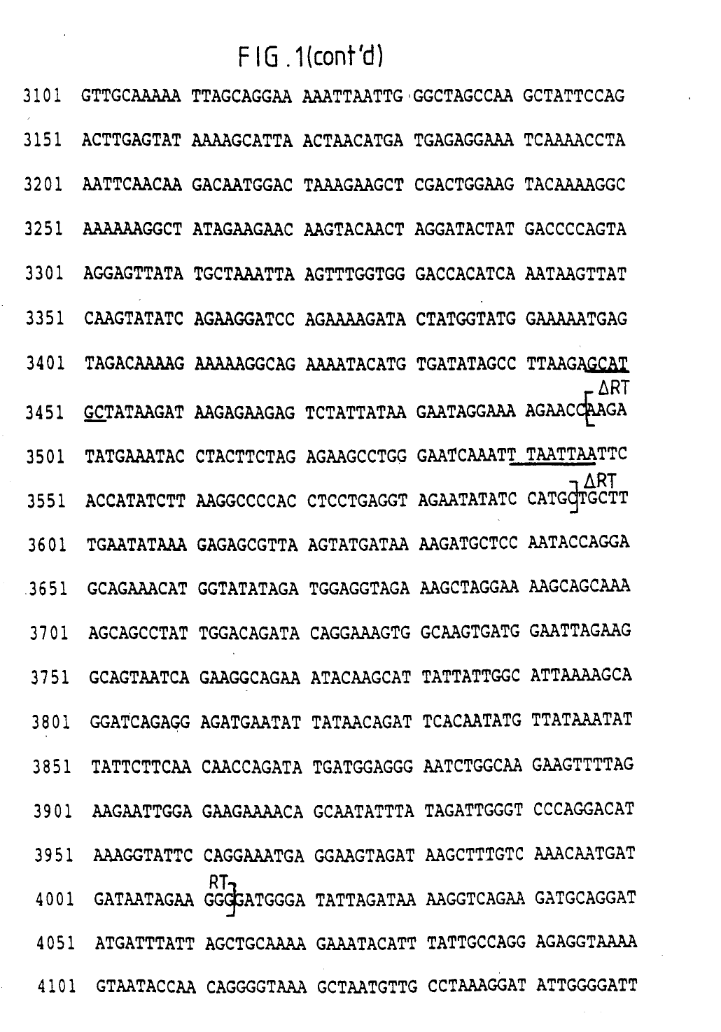

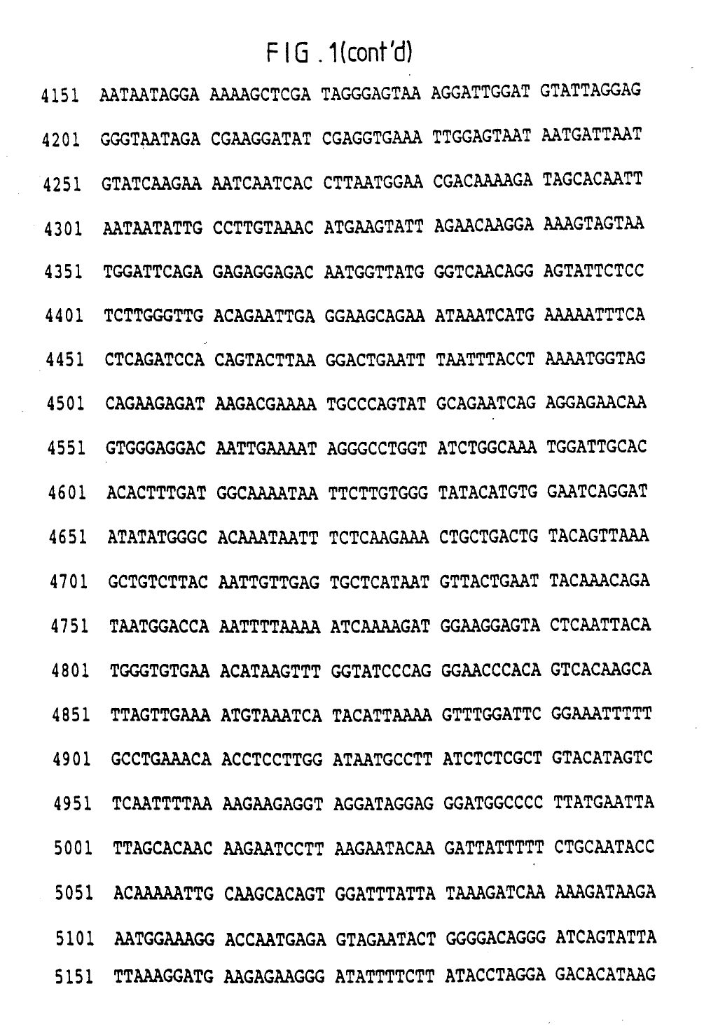

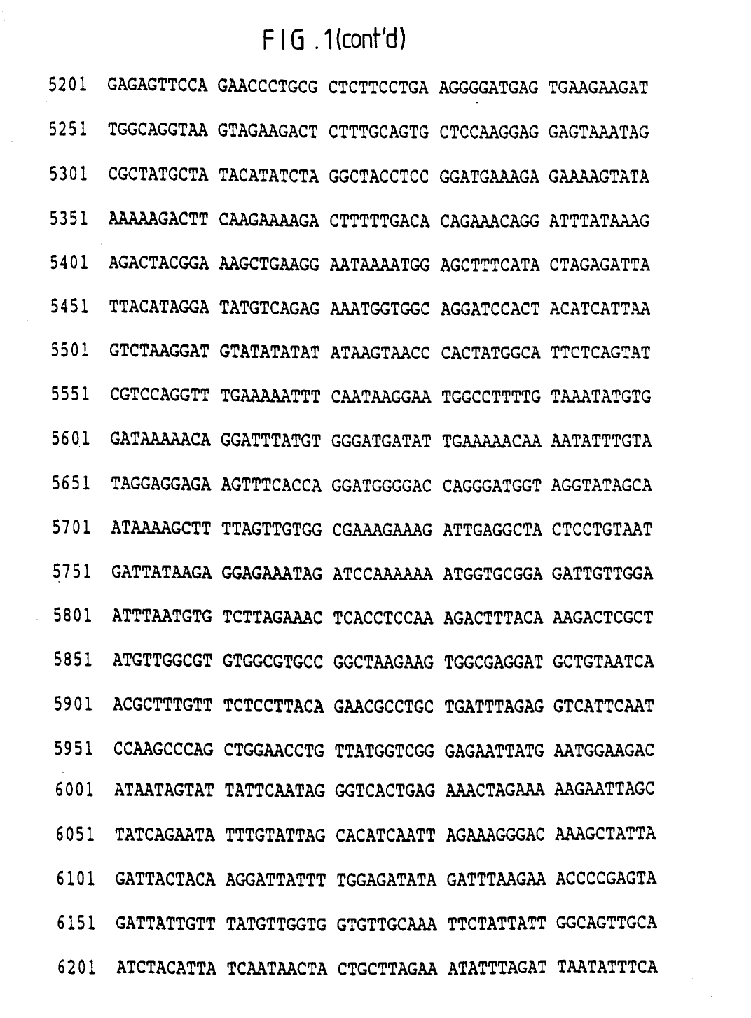

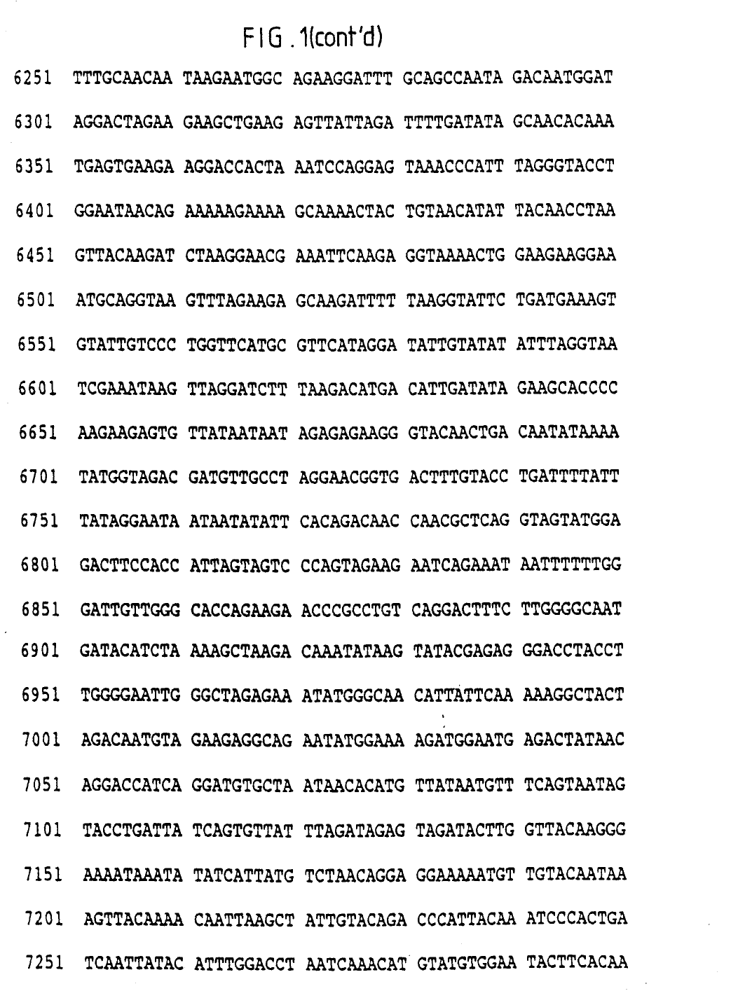

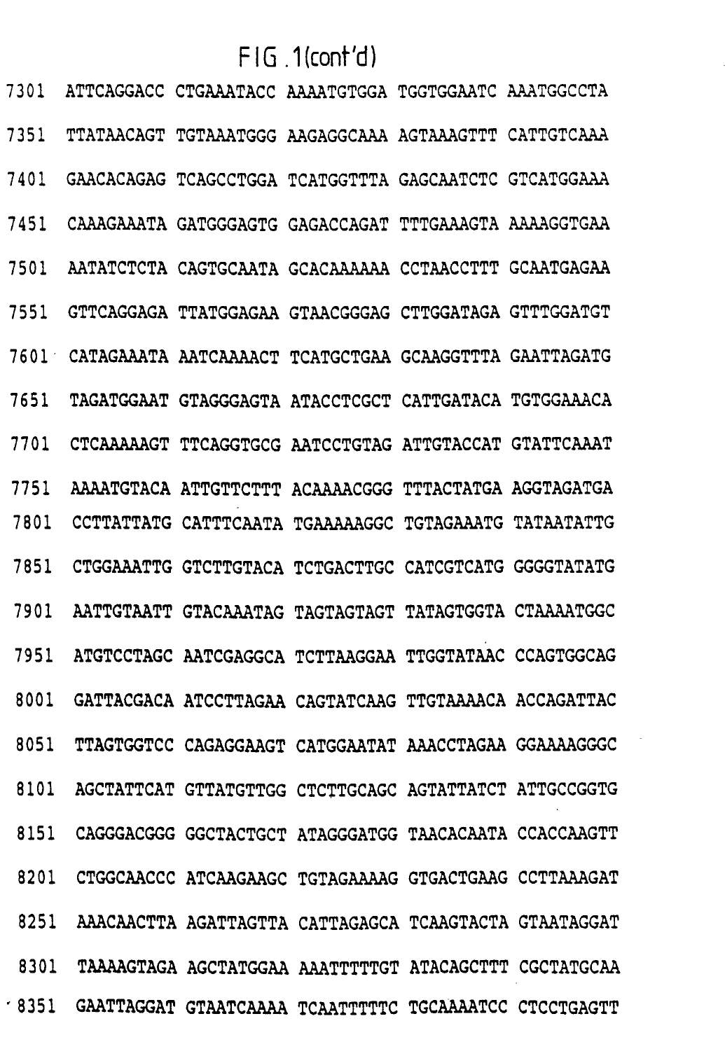

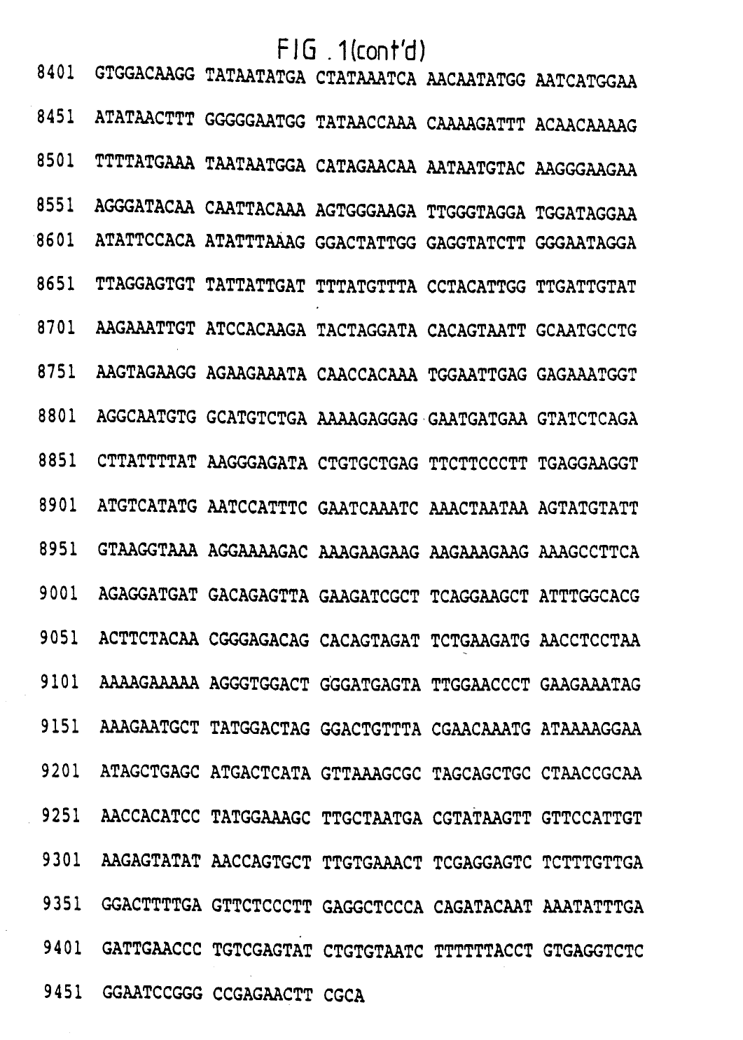

FIG. 1: Nucleotide sequence of FIV F14 (Petaluma strains) showing ΔRT site (3496 to 3595)(Sequence ID. No. 5) Pac I, Ncol and Sph I sites.

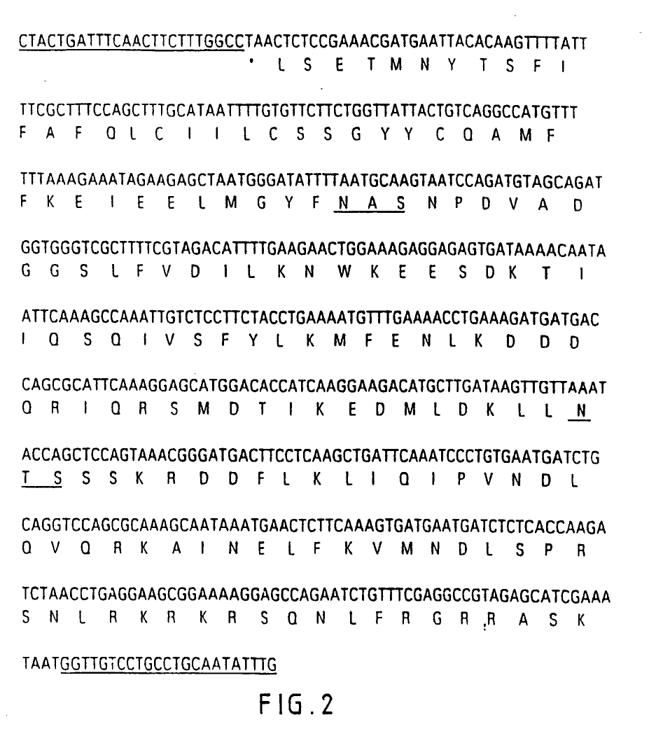

FIG 2: Feline γ-Interferon.

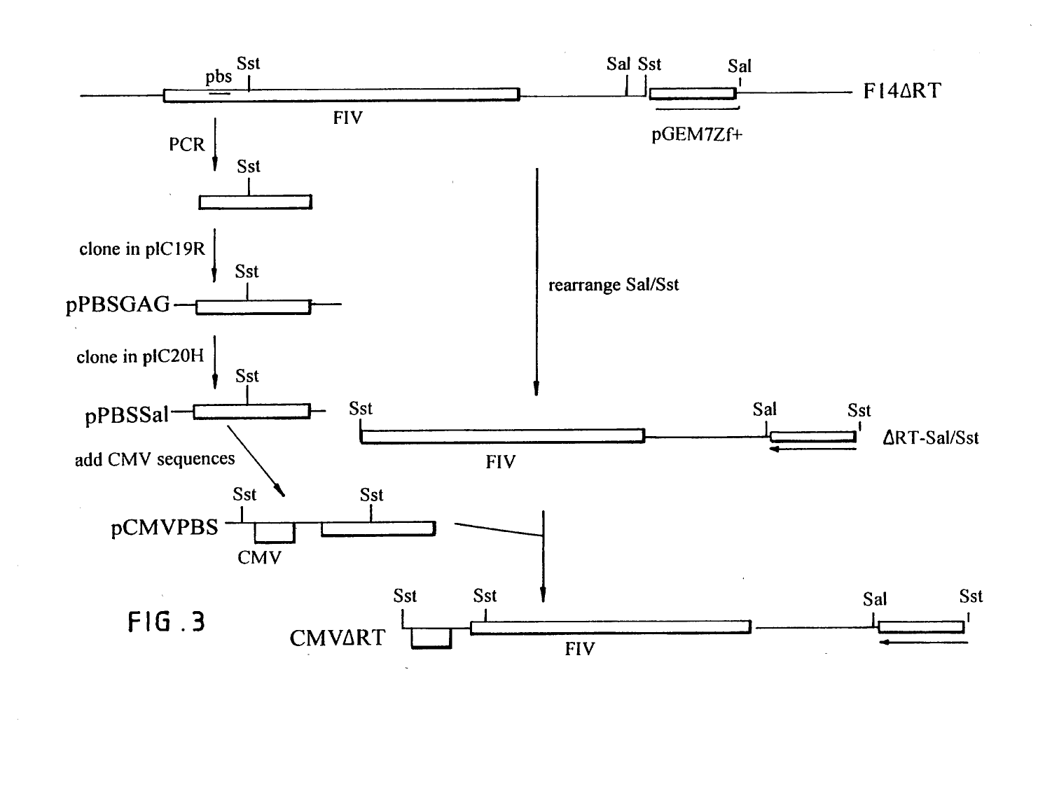

FIG. 3: Construction of CMVΔRT.

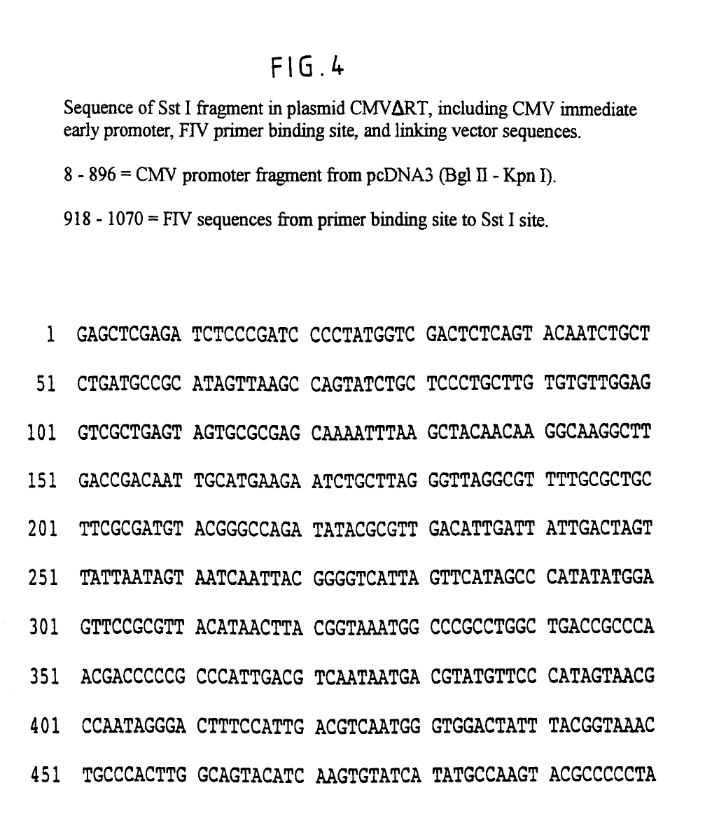

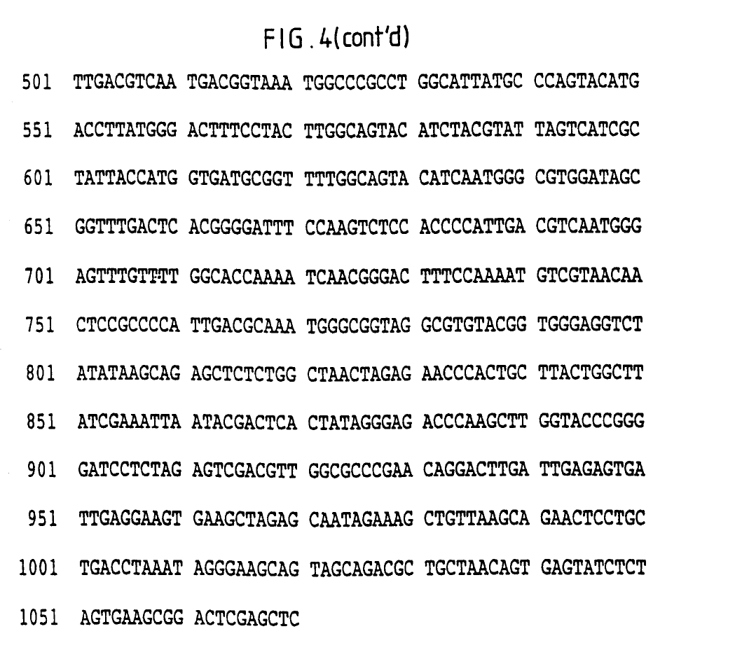

FIG. 4: Sequence of Sst I fragment in CMVΔRT (Sequence ID. No. 6).

FIG 5: Genome Map of FIV RT deletion mutant.

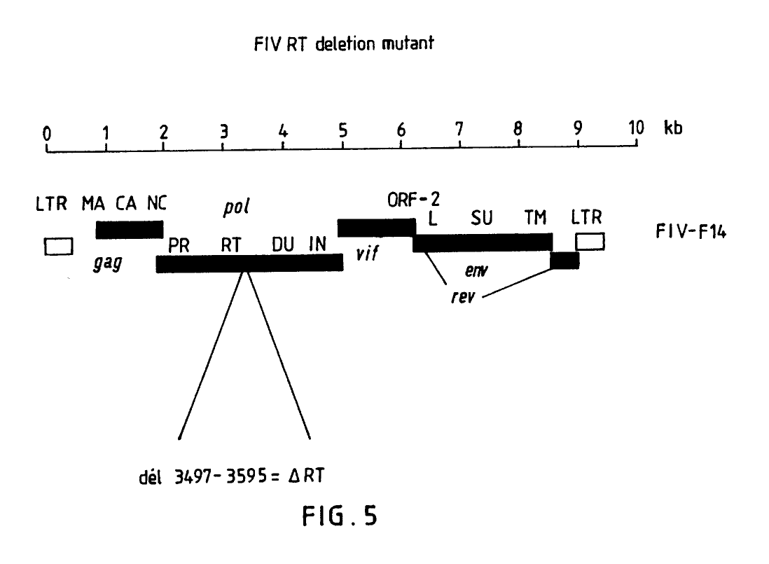

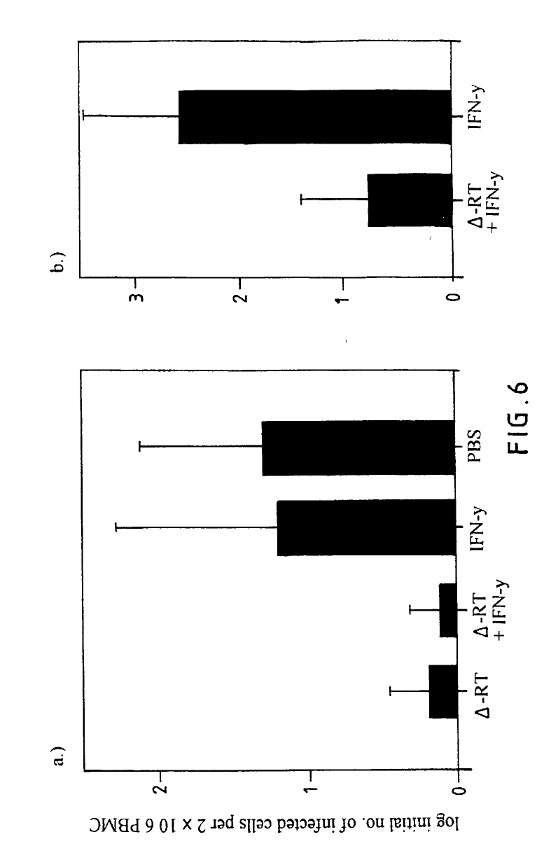

FIG 6: Peripheral blood viral loads in a) trial-6(a) at 7 weeks post challenge and in b) trial-6(b) at 6 weeks post challenge, expressed as the mean (+/−2SEM) of the log-transformed maximum likelihood estimates of the initial number of infected cells present in 2×10⁶ PBMC.

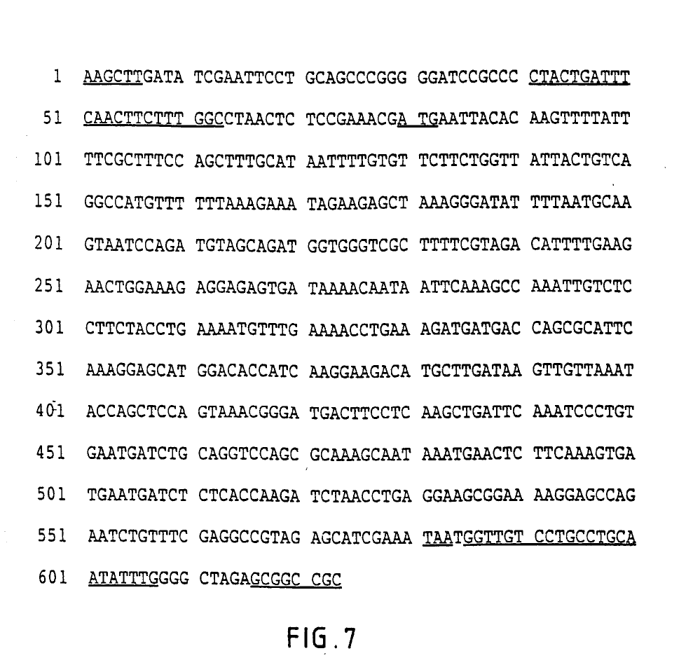

FIG. 7: Sequence of the Hind III—Not I fragment in plasmid pRSV-γ-IFN (Sequence ID. No. 7).

### EXAMPLES SECTION

### Derivation and Characterisation of a Defective FIV Provirus

Summary

The F14 clone of FIV-Petaluma was modified by introducing a deletion centred on a unique Pacl restriction site in the RT domain of the pol gene, in a region homologous to the “connection” domain of human immunodeficiency virus RT. A clone with a 33-codon, in-frame deletion was identified and designated FIV-ΔRT. This clone was characterised in vitro by transfection into fibroblasts. Following transfection: 1, syncytia were formed within 3 days; 2, cell lysates showed glycoprotein and Gag protein expression by Western blot; 3, antigen was pelleted from culture fluids by centrifugation at 100,000 ×g, suggesting it is in particulate form; 4, no RT activity above background was observed in the culture fluids; and 5, unlike cultures transfected with wild-type FIV-F14, no infectious virus was detected in the culture fluids.

### METHODS

1. Induction of FIV-Specific Cytotoxic T Cells

At 3, 6, 10, 12, 16 and 20 weeks post vector delivery and on the day of challenge, 5 ml peripheral venous blood was collected into an equal volume of Alsever's solution (Scottish Antibody Production Unit, Carluke, UK), and PBMC were prepared by centrifugation over Ficoll-Paque (Pharmacia LKB, Biotechnology Inc., Piscataway, N.J.) for the determination of virus-specific lymphocytoxicity. Fibroblast cell lines were derived from skin biopsy samples (4 mm in diameter) obtained from all cats under general anaesthesia prior to immunisation or challenge, and maintained in minimal essential medium (MEM) ALPHA medium with ribonucleosides and deoxyribonucleosides (Biological Industries, Paisley, UK) supplemented with 10% foetal bovine serum (FBS), 2 mM L-glutamine, and 100 IU of penicillin, 100 μg streptomycin, long of human epidermal growth factor (Sigma, Poole, UK) per ml.

Virus-specific effector CTL present in the fresh PBMC were detected using autologous or allogeneic skin fibroblast target cells labelled with 50 μCi of sodium [⁵¹Cr] chromate (Amersham International, Aylesbury, UK)/10⁶ cells for 18 hours at 37° C., washed three times, and then infected with 5 to 10 plaque-forming units/cell of recombinant vaccinia virus expressing either the gag or env gene product from FIV/Glasgow-14 or FIV/Petaluma, respectively, or with wild-type vaccinia virus for 1 hour at 37° C. Unbound virus was washed away, and the cells were incubated for an additional 2 hours to allow optimal expression of the FIV Gag and Env products. Standard microcytotoxicity assays were then performed in triplicate by adding appropriate numbers of effector cells to 1×10⁴ target cells to give effector: target (E:T) ratios of 50, 25, 12.5 and 6.25:1 as described previously (Flynn et al., (1996) supra).

2. Isolation of FIV

Peripheral blood mononuclear cells (PBMC) were isolated from heparinized venous peripheral blood by centrifugation over Ficoll-Hypaque (Pharmacia LKB, Biotechnology Inc., Piscataway, N.J.). Then 10⁶ PMBC were co-cultivated as described in Hosie M. J. and Flynn J. N. (1996) J. Virol. 70 pp. 7561-7568). Samples of culture supernatant were tested at intervals for the presence of FIV p24 by ELISA (IDEXX Laboratories, Portland, Me.) and cultures were maintained for 21 days before being scored as negative.

3. Quantitative Virus Isolation

The infectious virus burden was measured in peripheral blood mononuclear cells (PBMC) that had been isolated from heparinized peripheral blood by Ficoll-Paque separation (Pharmacia), frozen and stored under liquid nitrogen. Decreasing numbers of PBMC (2 ×10⁶, 2×10⁵, 2×10⁴, 2×10³, 2×10², 20 and 2) were co-cultivated in duplicate in 24-well plates with 5×10⁵ Miyazawa-1 cells in 1.5 ml RPMI-1640 medium (Gibco) supplemented with 10% foetal bovine serum (Imperial Laboratories), 2 mmol/l glutamine, 100 IU penicillin, 100 mg/ml streptomycin (all from Gibco BRL) and 5×10⁻⁵ mol/l 2-mercaptoethanol (Sigma Chemical Co.). Twice weekly, 0.5 ml of the culture supernatant was removed and replaced with fresh medium. The culture supernatant collected on day 14 was tested by ELISA for FIV p24 production (FIV antigen detection kit, IDEXX).

### Example 1

Construction of the Deletion in RT

The F14 clone of FIV/Petaluma (Olmsted et al. 1989 supra) which includes approximately 9 kb of uncharacterised feline genomic DNA flanking the proviral sequence within the vector pGEM-7Zf+(Promega) includes a unique Pac 1 site within the RT region of the pol gene (nucleotides 3540-3547). Linearised plasmid was purified by precipitation then digested with Bal31 exonuclease under conditions calculated to allow a rate of 30 bp/minute (Maniatis T. et al. supra). After purification by phenol/chloroform extraction and ethanol precipitation, exonuclease digested DNA was recircularised by ligation and the products were used to transform E.coli DS941 (Meaden et al. Gene (1994) Vol. 41 pp. 97-101). Clones were examined by polymerase chain reaction (PCR) amplification across a 235 bp region of pol encompassing the Pac 1 site. One clone (ΔRT) (Sequence ID. No. 5) with a large in-frame deletion 99bp was characterised by DNA sequencing using the PCR primers:

(Sequence ID. No.1)

| | |
|---|---|
| (1) | TGTGATATAGCCTTAAGAGC (3429-3448) |
| | and |

(Sequence ID No.2)

| | |
|---|---|
| (2) | TACCATGTTTCTGCTCCTGG (3645-3664) |

This clone was designated FIV-ΔRT (FIG. 1) (Sequence ID No. 5).

### Example 2

Characterisation of FIV-ΔRT

FIV-ΔRT (50 μg plasmid DNA) was transfected into CrFK cells by calcium phosphate co-precipitation. The parental F14 plasmid served as positive control. After 3 days, syncytia were observed in the transfected cultures but not in mock-transfected cells (no DNA). This result implied that cells expressing the deleted provirus were able to fuse with neighbouring cells, presumably because they elaborated functional envelope glycoprotein. Syncytia were readily stained by immunofluorescence using serum pooled from FIV-infected cats.

Production of viral proteins was also investigated by enzyme-linked immunosorbance assay (ELISA) and immunoblotting. Large amounts of Gag capsid protein (p24) were detected in culture supernatants 6 days after transfection with F14 or ΔRT (Table 1) commercial antigen ELISA (“Petcheck”; IDEXX Laboratories, USA). Other viral proteins in cell lysates were analysed by SDS PAGE and immunoblotting using serum pooled from FIV-infected cats. Gag precursor and mature (capsid) proteins, and also envelope surface glycoprotein, were observed.

The capsid antigen could be pelleted from cell supernatants by ultracentrifugation, as detected by ELISA and immunoblotting. Thus the defective provirus was still capable of directing synthesis of antigenic particles.

RT activity was measured in culture supernatants. Cultures corresponding to wild type F14 were strongly positive, whereas cells transfected with FIV-ΔRT showed no activity above background levels (Table 1).

The absence of infectious virus in the ΔRT cultures was confirmed by passage of cells or supernatant fluids to fresh CrFK cell monolayers. After 7 days, no syncytium formation, p24 antigen or RT activity was observed in cultures seeded with supernatant from ΔRT-transfected cells, whereas supernatant from cells transfected with wild-type FIV established infection rapidly. Occasional syncytia were observed in cultures seeded with ΔRT—transfected cells, presumably centred around individual transfected cells carried over from the initial exposure to DNA.

### Example 3

Construction of CMV-ΔRT

A region from the 5′ LTR to the primer binding site in F14ΔRT was replaced by the immediate early promoter from human cytomegalovirus. This procedure was designed both to enhance expression of FIV antigens, and to reduce the risk of reversion to a replicating provirus, in tissues after inoculation of DNA. The construct was designated CMVΔRT, and its construction was achieved as follows:

Restriction sites for endonucleases Sal I and Sst I were mapped. F14ΔRT was rearranged as in FIG. 3 to an intermediate (designated ΔRT-Sal/Sst) having a unique Sst I site. Accordingly, Sal I and Sst I were used to digest plasmid F14ΔRT, the resulting mixture of fragments was religated and used to transform E.coli (DS941), and a clone with the structure expected of ΔRT-Sal/Sst was identified. CMV sequences could then be introduced upstream of the Sst I site.

A PCR product encompassing FIV sequences from the primer binding site to a point downstream of the Sst I site was derived from the F14 plasmid using Taq polymerase (Perkin Elmer) and the method of Saiki et al (1985) Science 230 pp. 1350-1354; The primers used (corresponding to co-ordinates 356-376 (Sequence ID No. 3) and 1963-1980 (Sequence ID No. 4) of the F14 provirus) were constructed with additional Sal I “tails”, and had the sequences: GATCGTCGACGTTGGCGCCCGAACAGGACT (5′) and GATCGTCGACTTATAAATCCAATAGTTT (3′). This PCR product was cloned into the Hinc II site of plasmid vector pIC19R (Marsh et al. (1984) Gene 32 pp 481-485) to yield pPBSGAG. FIV sequence from pPBSGAG was then released as a Sal I fragment and cloned into the Sal I site of pIC20H (Marsh et al. supra) to give pPBSSal. The CMV IE promoter was cloned infront of these FIV sequences as a Bgl II-Kpn I fragment from expression vector pcDNA3 (Invitrogen), yielding pCMVPBS. An Sst I fragment from this clone, including the IE promoter and FIV sequences from the primer binding site to the proviral Sst I site, was then cloned into the Sst I site in ΔRT-Sal/Sst. The resulting DNA sequence from within the CMV IE promoter to a point downstream of the FIV proviral Sst I site was confirmed by direct sequencing.

The sequence of the Sst I fragment in CMVΔRT is shown in FIG. 4 (Sequence ID. No. 6). FIV sequences downstream of the Sst I site are identical to those in F14ΔRT.

### Example 4

Construction of pRSV-γ-IFN

Feline γ-interferon cDNA was available as a cDNA clone in pCR-ScriptSK(+) (Stratagene) as described in Argyle D. J. et al. (1995) (DNA Sequence 5, 169-171). The cDNA sequence was excised with restriction enzymes HindIII and NOtI (Sequence ID No. 7) and inserted into pRc/RSV expression vector (Invitrogen) to produce the pRSV-γIFN plasmid.

### Example 5

FIV DNA Immunisation Trial: Protection of Vaccinated Cats

Procedure

The efficacy of DNA immunisation to protect cats from infection with feline immunodeficiency virus (FIV) was determined. Twenty 12 week old kittens were randomised into 4 groups of 5. The DNA used in the inoculations comprised a plasmid ΔRT, either alone or in conjunction with feline γ-IFN DNA, as shown below:

| Group No. | Cat No. | Plasmid |
|---|---|---|
| Group 1 | A481-485 | 100 μg ΔRT |
| Group 2 | A486-490 | 100 μg ΔRT + 100 μg pRSV-γ- |
| | | IFN |
| Group 3 | A491-495 | 100 μg pRSV-γ-IFN |
| Group 4 | A496-500 | no DNA |

The cats were inoculated intramuscularly with test DNA at each of 4 sites with 100μg DNA in 200μl PBS on weeks 0, 10 and 23. The cats were challenged intraperitoneally on week 26 with 25 cat infectious doses 50% (CID₅₀) of FIV-Petaluma derived from the F-14 molecular clone, propagated in Q201 cells (Willett et al. (1991) AIDS Vol. 5 pp. 1469-1475).

Results

Antibody responses were measured by immunoblotting according to the method of Hosie M. J., O. Jarrett (1990) AIDS 4 pp. 215-220 and to peptides representing two immunodominant epitopes from the viral envelope proteins (V3 and TM) by enzyme linked immunosorbent assay (ELISA) (Hosie M. J. and Flynn J. N., (1996) J. Virol. 70 pp. 7561-7568) 3 weeks after each vaccination and 3, 6, 9, and 12 weeks following challenge.

Assays for cytotoxic T cell (CTL) activity against FIV Env and Gag proteins were conducted during the immunisation schedule and at the day of challenge (Hosie M. J. and Flynn J. N. (1996) supra).

Antibody Responses

No antibodies were detected by peptide ELISA (as above) prior to the day of challenge. Following challenge, any antibody responses could therefore be equated with infection. The results are included in Table 2.

Cytotoxic T Cell Response (CTL Responses)

FIV Gag- and Env-specific effector CTL activity was detected following the method of Hosie M. J. and Flynn J. N. (1996) supra, in the fresh peripheral blood of all cats immunised with the ΔRT plasmid (A481-A485) three weeks following vector delivery. The response was only observed on autologous target cells, suggesting that the response was MHC-restricted. Furthermore, there was no recognition of target cells infected with the wild-type vaccinia virus confirming the specificity of the response. The FIF Gag-specific responses appeared higher than (A481 and A482) or similar to the levels of Env- specific lysis observed at an E:T ratio 50:1 and levels ranged between 20 and 54%. This pattern of responses is similar to that observed in the peripheral blood of cats immunised with inactivated whole virus vaccine based on the FL4 cell line. However, the levels of specific lysis observed with WIV inactivated virus vaccines are generally slightly lower than those detected in the present study with the ΔRT plasmid, and the predominant CTL response is directed towards Env rather than Gag (Flynn et al., (1995) Aids Res. Human Retro. 11 pp. 1107-1113, Hosie and Flynn, (1996) supra).

Co-immunisation with the ΔRT plasmid and a feline γ-IFN plasmid induced very high levels (up to 73% specific lysis) of Gag-specific lysis in 3 out of 5 vaccinated cats (A486, A488 and A490), and Env-specific lysis in 2 out of 5 cats (A487 and A489). However, this response did not appear to be entirely MHC-restricted since considerable lysis of allogeneic target cells was also observed. The non-specific nature of the cytolytic responses observed was further confirmed by the recognition of autologous target cells infected with wild-type vaccinia virus, in 3 out of 5 cats. Immunisation with the γ-IFN plasmid alone resulted in the induction of FIV-specific cytolytic responses in 3 out of 5 cats (A491 to A493), in either autologous or allogeneic target cells. In addition, high levels of lysis were observed in 2 cats (A492 and A493) using target cells infected with wild-type vaccinia virus. These results suggest that in vivo delivery of the feline γ-IFN plasmid to cats may elicit non-specific cellular immune responses such as NK-type activity.

No FIV-specific immune responses were detected in control cats immunised with PBS alone.

By 6 weeks after vector delivery, significant levels (>10% specific lysis) of FIV Gag-specific CTL activity was detectable in 4 out of 5 cats immunised with the ΔRT plasmid, and 3 of these cats also had significant levels of Env-specific CTL activity. However, the levels detected were lower than those observed at 3 weeks post immunisation. In the group immunised with ΔRT and γ-IFN plasmids, no FIV-specific CTL activity was detected. Likewise no CTL activity was detected in the control groups immunised with γ-IFN alone or with PBS, the one exception being A491 which displayed a response to FIV Gag and Env.

At 10 weeks post immunisation the CTL responses detected in the group immunised with ΔRT had declined still further, with FIV Gag-specific activity detectable in one cat (A484) and Env-specific activity in another (A482). At this time Gag-specific lysis was observed in 2 cats immunised with ΔRT together with γ-IFN and Env-specific activity was observed in A490. However the levels observed were rather low compared to those at the 3 week time point. Again no activity was observed in control cats. The cats were re-boosted at this time and the FIV-specific CTL responses induced the peripheral blood analysed 2 weeks later.

The boost at week 10 had the effect of raising the FIV Gag-specific CTL activity in 3 out of 5 cats immunised with the ΔRT construct, in addition non-specific responses were detected in 2 cats. A similar effect was noted in cats immunised with ΔRT and γ-IFN, with Gag-specific CTL activity boosted in 2 cats. A490 maintained similar levels of Env-specific lysis to that observed at week 10. Negligible FIV-specific lysis was recorded in control cats.

Assays performed at weeks 16 and 20 were unremarkable, and assays performed on the day of challenge with 25 CID₅₀ of F14 FIV/Petaluma, revealed low levels (12-15% specific lysis) of Gag-specific CTL activity in 2/5 ΔRT immunised cats and negligible activity in the cats immunised with ΔRT and γ-IFN.

Results of Virus Detection

Virus isolation from PBMC was attempted following immunisation but was negative at all times prior to and including the day of challenge, indicating that there was no reversion to virulence of the mutant provirus during this period. Following challenge, cats were monitored for infection by virus isolation. By 9 weeks post challenge, 5/5 control cats receiving no DNA had become infected, together with 5/5 cats inoculated with feline γ-IFN DNA. In contrast, there was evidence of protection in the groups inoculated with ΔRT DNA (Table 3). No virus could be isolated from one of the 5 cats in group 1 or from 3/5 cats in group 2. Furthermore, the viral loads measured by quantitative co-culture of PBMC with MYA cells in the infected cats that had been inoculated with ΔRT were lower than those of the cats in the two control group (Table 4).

Since several parameters that were measured gave an indication of infection and viral load following challenge, a clinical scoring system was adopted in order to compare the outcomes between groups (Table 5a). Clinical scores were significantly lower in the groups immunised with ΔRT and ΔRT+γ-IFN compared to their appropriate control (p<0.05 and 0.005 respectively, Table 5b), providing further evidence that FIV DNA immunisation induced protective immunity that was augmented by feline γ-IFN DNA.

### Example 6

Shortened FIVΔRT Immunisation Schedule

To investigate whether the earlier described immunisation schedule could be reduced without compromising protection, a second experiment was conducted in which 2 groups of 5 cats received either FIVΔRT+IFN-γ or IFN-γ alone at 0,4 and 8 weeks. As in the first trial, this regimen induced broad spectrum cytolytic activity but no detectable antibody responses using the same series of assays. After challenge at 12 weeks, 2/5 vaccinates remained seronegative and virus could not be isolated at any of the times tested (Table 6(a) and 6(b)) whereas all of the IFN-γ alone controls became seropositive and positive by virus isolation, consistent with the results of the first trial. Again, immunoblot analysis corroborated these findings fully. Quantitative measurements of virus in the second trial (FIG. 6) revealed that at 6 weeks post challenge, the FIVΔRT+IFN-γ vaccinates developed significantly lower viral loads compared to the IFN-γ vaccinates (P=0.027).

TABLE 1
Production of p24 but not RT by ΔRT DNA

| | |
|---|---|
| | Post supernatant |
| Post transfection | transfer |

| | p24 | | p24 | |
|---|---|---|---|---|
| DNA | (OD₄₀₅) | RT | (OD₄₀₅) | RT |
| F14 | >3.00 | 255 | >3.00 | 2329 |
| ΔRT | 1.07 | 98 | 0 | 86 |
| Control | 0.11 | 87 | 0 | 91 |

TABLE 2
Results of assays for virus infection post-challenge weeks post challenge

| | | 6w¹ | | | | | | | | | | | |
|---|---|---|---|---|---|---|---|---|---|---|---|---|---|
| | | | | PCR | | 7w² | 9w | | | | 12w² | | |
| | Cat no. | α-TM | blot | pol | VI | VI | α-TM | α-V3 | blot | VI | α-TM | blot | VI |
| ΔRT | A481 | 0 | − | + | + | + | 0 | 0 | + | nd | 0 | + | − |
| | A482 | 5 | − | + | + | − | 5 | 0 | + | − | 0 | + | − |
| | A483 | 0 | − | + | + | + | 0 | 0 | + | nd | 0 | + | + |
| | A484 | 0 | − | − | − | − | 0 | 0 | − | − | 0 | − | − |
| | A485 | 125 | + | + | + | + | 125 | 5 | + | nd | 25 | + | − |
| ΔART + | A486 | 5 | − | * | − | − | 0 | 0 | − | − | 0 | − | − |
| pRSV- | A487 | 0 | (+) | + | + | + | 25 | 0 | + | nd | 0 | + | − |
| IFN-γ | A488 | 0 | − | − | − | − | 0 | 0 | − | − | 0 | − | − |
| | A489 | 5 | (+) | + | + | + | 125 | 5 | + | nd | 125 | + | − |
| | A490 | 0 | − | − | − | − | 0 | 0 | − | − | 0 | − | − |
| pRSV- | A491 | 125 | + | + | + | + | 625 | 5 | + | nd | 125 | + | + |
| IFN-γ | A492 | 5 | + | + | + | + | 625 | 0 | + | nd | 25 | + | − |
| | A493 | 25 | + | + | + | + | 125 | 0 | + | nd | 25 | + | + |
| | A494 | 25 | + | + | + | + | 125 | 0 | + | nd | 25 | + | + |
| | A495 | 5 | + | + | + | + | 25 | 0 | + | nd | 5 | + | + |
| no | A496 | 25 | + | + | + | + | 125 | 0 | + | nd | 25 | + | + |
| DNA | A497 | 25 | + | + | + | + | 25 | 0 | + | nd | 5 | + | + |
| control | A498 | 5 | + | + | + | + | 125 | 0 | + | nd | 5 | + | nd |
| | A499 | 25 | + | + | + | + | 125 | 0 | + | nd | 125 | + | + |
| | A500 | 25 | + | + | + | + | 25 | 0 | + | nd | 5 | − | + |

¹quantitative PCR data available
²quantitative virus isolation data available
*Indeterminate value

TABLE 3
Protection against FIV infection induced by DNA immunisation

| | | Proportion |
|---|---|---|
| Group | Inoculum | protected |
| 1 | ΔRT | 1/5 |
| 2 | ΔRT + γ-IFN | 3/5 |
| 3 | γ-IFN | 0/5 |
| 4 | PBS | 0/5 |

TABLE 4
Results of Quantiative Virus Isolation Number of PBMC Plated

| DNA | Cat No. | 2 × 10⁶ | 2 × 10⁵ | 2 × 10⁴ |
|---|---|---|---|---|
| RT | A481¹ | 1/2 | 0/2 | 0/2 |
| | A482¹ | 1/2 | 0/2 | 0/2 |
| | A483¹ | 0/2² | 0/2 | 0/2 |
| | A484¹ | 0/1 | 0/2 | 0/2 |
| | A485¹ | 0/2 | 0/2 | 0/2 |
| RT + γIFN | A486¹ | 0/2 | 0/2 | 0/2 |
| | A487 | 1/2 | 0/2 | 0/2 |
| | A488 | 0/2 | 0/2 | 0/2 |
| | A489¹ | 0/2 | 0/2 | 0/2 |
| | A490¹ | 0/2 | 0/2 | 0/2 |
| γ-IFN | A491 | 2/2 | 1/2 | 0/2 |
| | A492¹ | 0/2 | 0/2 | 0/2 |
| | A493¹ | 2/2 | 1/2 | 0/2 |
| | A494¹ | 0/1 | 0/2 | 0/2 |
| | A495 | 2/2 | 1/2 | 0/2 |
| None (PBS) | A496¹ | 2/2 | 1/2 | 0/2 |
| | A497 | 2/2 | 1/2 | 0/2 |
| | A498 | 2/2 | 0/2 | 0/2 |
| | A499¹ | 2/2 | 0/2 | 0/2 |
| | A500 | nd | 0/1 | 0/2 |

¹× 10⁶ cells available for test
²1/2 wells near cut off OD
nd = not done

TABLE 5
Ranking of results by clinical score
a. Clinical Score Ratings

| Virus isolation | |
|---|---|
| positive at 3 weeks pc | 1 |
| positive at 6 weeks pc | 1 |
| Immunoblot analysis of plasma pc | |
| positive at 6 weeks pc | 1 |
| positive at 9 weeks pc | 1 |
| Viral load quantiation | |
| virus isolated from 2 × 10⁶ PBMC | 1 |
| virus isolated from 2 × 10⁵ PBMC | 1 |
| virus isolated from 2 × 10⁴ PBMC | 1 |
| Possible maximum score | 7 |

b. Clinical Scores of Cats following challenge

| | | | |
|---|---|---|---|
| Group 1 | | Group 3 | |
| ΔRT | | γIFN | |
| A481 | 3 | A491 | 6 |
| A482 | 4 | A492 | 4 |
| A483 | 2 | A493 | 5 |
| A484 | 0 | A494 | 3 |
| A485 | 4 | A495 | 6 |
| mean | 2.6¹ | | 4.8 |
| SEM | 0.75 | | 0.58 |
| Group 2 | | Group 4 | |
| ΔRT + γIFN | | PBS | |
| A486 | 0 | A496 | 5 |
| A487 | 4 | A497 | 6 |
| A488 | 0 | A498 | 4 |
| A489 | 3 | A499 | 4 |
| A490 | 0 | A500 | 4 |
| mean | 1.4² | | 4.6 |
| SEM | 0.87 | | 0.4 |

¹P = 0.0462
²P = 0.0103

TABLE: 6(a)
weeks post challenge

| DNA | | 0 | | | 3 | | 6 | | 9 | | 12 | | |
|---|---|---|---|---|---|---|---|---|---|---|---|---|---|
| inoculum | Cat | IB | VI | TM | IB | VI | IB | VI | IB | VI | IB | VI | TM |
| FIVΔRT | 1 | − | − | 0 | − | − | − | + | + | + | + | − | 0 |
| | 2 | − | − | 0 | − | + | − | + | + | + | + | − | 0 |
| | 3 | − | − | 0 | − | − | − | + | + | + | + | + | 0 |
| | 4 | − | − | 0 | − | − | − | − | − | − | − | − | 0 |
| | 5 | − | − | 0 | − | + | + | + | + | + | + | − | 25 |
| FIVΔRT + | 1 | − | − | 0 | − | − | − | − | − | − | − | − | 0 |
| IFN-γ | 2 | − | − | 0 | − | − | + | + | + | + | + | − | 0 |
| | 3 | − | − | 0 | − | − | − | − | − | − | − | − | 0 |
| | 4 | − | − | 0 | − | − | + | + | + | + | + | − | 125 |
| | 5 | − | − | 0 | − | − | − | − | − | − | − | − | 0 |
| IFN-γ | 1 | − | − | 0 | − | + | + | + | + | + | + | + | 125 |
| | 2 | − | − | 0 | − | + | + | + | + | + | + | − | 25 |
| | 3 | − | − | 0 | − | − | + | + | + | + | + | + | 25 |
| | 4 | − | − | 0 | − | − | + | + | + | + | + | + | 25 |
| | 5 | − | − | 0 | − | + | + | + | + | + | + | + | 5 |
| no DNA | 1 | − | − | 0 | − | − | + | + | + | + | + | + | 25 |
| | 2 | − | − | 0 | − | + | + | + | + | + | + | + | 5 |
| | 3 | − | − | 0 | − | − | + | + | + | + | + | + | 5 |
| | 4 | − | − | 0 | − | − | + | + | + | + | + | + | 125 |
| | 5 | − | − | 0 | − | + | + | + | + | + | + | + | 5 |

IB: immunoblot
VI: virus isolation
TM: titre of antibodies recognizing TM peptide

TABLE: 6(b)
weeks post challenge

| DNA | | 0 | | | 3 | | 6 | | 9 | | 12 | | |
|---|---|---|---|---|---|---|---|---|---|---|---|---|---|
| inoculum | Cat | IB | VI | TM | IB | VI | IB | VI | IB | VI | IB | VI | TM |
| FIVΔRT + | 1 | − | − | 0 | nd | + | + | + | nd | + | + | + | 25 |
| IFN-γ | 2 | − | − | 0 | nd | − | − | − | nd | − | − | − | 0 |
| | 3 | − | − | − | nd | − | − | + | nd | + | + | − | 0 |
| | 4 | − | − | 0 | nd | + | + | + | nd | + | + | − | 25 |
| | 5 | − | − | 0 | nd | − | − | − | nd | − | − | − | 0 |
| IFN-γ | 1 | − | − | 0 | nd | + | + | + | nd | + | + | + | 25 |
| | 2 | − | − | 0 | nd | + | + | + | nd | + | + | − | 125 |
| | 3 | − | − | 0 | nd | + | − | + | nd | + | + | + | 5 |
| | 4 | − | − | 0 | nd | − | − | + | nd | + | + | + | 25 |
| | 5 | − | − | 0 | nd | − | − | + | nd | + | + | + | 25 |

IB: immunoblot
VI: virus isolation
TM: titre of antibodies recognizing TM peptide
nd: not done

1. A vaccine formulation comprising a FIPV polynucleotide comprising a dysfunctional pol gene which is substantially incapable of encoding a functionally competent RT or a functional RT fragment thereof.

2. A formulation according to claim 1 wherein the FIPV polynucleotide comprises a deletion within the RT domain of the pol gene.

3. A formulation according to claim 1 or claim 2 wherein the deletion within the RT domain of the pol gene is an in-frame deletion.

4. A formulation according to any one of the preceding claims further comprising a polynucleotide fragment encoding a cytokine.

5. A formulation according to claim 4 wherein the polynucleotide fragment encoding the said cytokine is located within an in-frame deletion site within the RT domain of the pol gene.

6. A formulation according to claim 4 or claim 5 wherein the cytokine is feline interferon-γ.

7. A formulation according to any one of claims 1 to 6 wherein the FIPV polynucleotide comprises a deletion located at a restriction enzyme site unique to the RT domain of the pol gene.

8. A formulation according to claim 7 wherein the FIPV polynucleotide comprises a deletion located at a restriction enzyme site selected from Nco1, Pac1 and Sph1.

9. A formulation according to any one of the preceding claims wherein the FIPV polynucleotide is in naked form.

10. A formulation according to any one of claims 1 to 8 wherein the FIPV polynucleotide fragment is in the form of a vector.

11. A formulation according to any preceding claim further comprising an adjuvant.

12. A vaccine formulation according to any one of claims 1 to 9 and 11 wherein the FIPV polynucleotide is in the form of a salt.

13. A FIPV polynucleotide fragment which is substantially incapable of encoding a functional RT or a functional RT fragment thereof for use as a medicament for FIV-related disease.

14. A FIPV polynucleotide fragment comprising a deletion within the RT domain of the pol gene for use as a medicament for FIV-related disease.

15. A FIPV polynucleotide fragment comprising an in-frame deletion within the RT domain of the pol gene for use as a medicament for FIV-related disease.

16. A polynucleotide fragment according to any one of claims 13 to 15 further comprising a polynucleotide fragment encoding a cytokine for use as a medicament for FIV-related disease.

17. A polynucleotide fragment according to claim 16 wherein the polynucleotide encoding a cytokine is located within an in-frame deletion site of the polynucleotide fragment encoding a FIPV, for use as a medicament for FIV-related disease.

18. Use of a FIPV comprising a dysfunctional pol gene in the manufacture of a vaccine for the prophylaxis and/or therapy of FIV-related disease.

19. Use of a FIPV according to claim 18 wherein the pol gene comprises a deletion within its RT domain.

20. Use according to claim 18 or claim 19 wherein the pol gene comprises an in-frame deletion within its RT domain.

21. Use according to any one of claims 18 or 20 wherein the pol gene comprises a deletion located at an enzyme restriction site selected from Pacl, Nco1 and Sph1.

22. A method of vaccinating against FIV-related disease in a mammal which comprises administering to the mammal an effective, non-toxic amount of a vaccine formulation according to any one of claims 1-12 or a polynucleotide fragment according to any one of claims 24-26.

23. A method according to claim 22 wherein the vaccine formulation comprises an FIPV fragment comprising an in-frame deletion within the RT domain of the pol gene.

24. A FIPV polynucleotide fragment comprising an in-frame deletion and/or insertion therein in the RT region of the RT domain of the pol gene.

25. A polynucleotide fragment according to claim 24 comprising an in-frame insertion therein comprising at least one nucleotide in the RT region of the RT domain of the pol gene.

26. A FIPV polynucleotide fragment according to claim 24 or claim 25 wherein the at least one nucleotide is a further polynucleotide fragment encoding for a cytokine in an in-frame deletion site of the RT domain of the pol gene.

27. A polynucleotide fragment according to any one of claims 24 to 26 wherein the cytokine is feline interferon-γ.
